(19) United States
(12) Patent Application Publication
(10) Pub. No.: US 2023/0092990 A1
(43) Pub. Date: Mar. 23, 2023

(54) MARKER AND PRODUCT FOR AUXILIARY DIAGNOSIS OF VALVULAR HEART DISEASE (VHD)

(71) Applicant: GUANGDONG BEATING ORIGIN REGENERATIVE MEDICINE CO., LTD., Foshan (CN)

(72) Inventors: Bin LIN, Foshan (CN); Rong ZENG, Foshan (CN); Weiwei KONG, Foshan (CN)

(73) Assignee: GUANGDONG BEATING ORIGIN REGENERATIVE MEDICINE CO., LTD., Foshan (CN)

(21) Appl. No.: 17/994,029

(22) Filed: Nov. 25, 2022

Related U.S. Application Data
(63) Continuation of application No. PCT/CN2021/116405, filed on Sep. 3, 2021.

(30) Foreign Application Priority Data
May 25, 2021 (CN) 202110572277.1

(51) Int. Cl. G01N 33/68

(52) U.S. Cl. CPC G01N 33/6887; G01N 2800/32; G01N 2333/70596

(57) ABSTRACT

A marker and product for auxiliary diagnosis of valvular heart disease (VHD) is provided. The present disclosure provides for the first time using PLAUR as a biomarker for auxiliary diagnosis of VHD. When the PLAUR is used for auxiliary diagnosis, the diagnostic result can be obtained in only one working day (high detection speed) with sensitivity/accuracy is much higher than the sensitivity/accuracy of NT-ProBNP and hsCRP, and a high-throughput operation is enabled.

### CROSS REFERENCE TO THE RELATED APPLICATIONS

This application is a continuation application of International Application No. PCT/CN2021/116405, filed on Sep. 3, 2021, which is based upon and claims priority to Chinese Patent Application No. 202110572277.1, filed on May 25, 2021, the entire contents of which are incorporated herein by reference.

### TECHNICAL FIELD

The present disclosure belongs to the technical field of biomedicine and, in particular, relates to a marker and product for auxiliary diagnosis of valvular heart disease (VHD).

### BACKGROUND

VHD refers to the anatomical and functional abnormalities of one or more valves caused by congenital developmental malformations or acquired lesions, which result in valvular stenosis and/or regurgitation, mainly including aortic stenosis (AS) or aortic regurgitation (AR) and mitral stenosis (MS) or mitral regurgitation (MR).

Cardiac valve stenosis or regurgitation directly affects the normal blood flow in the heart, increases a burden on the heart, causes damage to the cardiac function, and eventually leads to heart failure and death of a patient. VHD is a common cardiovascular disease (CVD) that endangers human life and health, and an incidence of VHD gradually increases with age. According to research reports, the incidence of VHD is low (less than 2%) in the young population and is significantly increased in the elderly population, and the incidence of VHD is increased to 13.2% in a population at an age of 75 or higher. With increased lifespan and exacerbated aging, the number of elderly VHD patients in China is gradually increasing. Due to poor immune systems and diminished faculties of elderly patients, complications are likely to occur, and thus, treatment of elderly patients has high risk and may result in death, which brings heavy family and social burdens.

The timely, accurate, and rapid diagnosis and early treatment is the most effective way to control the progression of VHD. At present, the diagnosis of VHD is mainly based on cardiac color Doppler ultrasound. Although this method is the most informative, there may be blind areas, and factors such as severe emphysema, thoracic deformity, and severe obesity may affect the diagnosis. Therefore, other indexes are required for auxiliary diagnosis. At present, B-type natriuretic peptide (BNP) is the only recognized biomarker for VHD diagnosis, which is mainly synthesized in the heart and is regulated by a myocardial pressure. BNP is currently the most widely used marker for cardiomyopathy. In the 2017 Guidelines for the Management of Valvular Heart Disease issued by the European Society of Cardiology (ESC) and the European Association for Cardio-Thoracic Surgery (EACTS), serum BNP is specified as a severity and prognostic marker for AS and MR. However, so far, the threshold of the marker has not been clearly defined, and there has been no clear recommendation for its use. In addition, other conditions (such as airway disease, obesity, atrial fibrillation (AF), liver cirrhosis, and age difference) may also cause changes in the BNP level, which limits the application of BNP in the evaluation of individual patients. The development and application of new biomarkers will bring new breakthroughs for VHD diagnosis.

The urokinase-type plasminogen activator receptor (uPAR, also known as PLAUR) is a glycoprotein with a molecular mass of 55 kD to 60 kD and includes 313 amino acid residues. Through disulfide bonds, the amino acid residues constitute three homologous folding domains, which are named DI, DII, and DIII in sequence from the N-terminus. PLAUR does not include a transmembrane sequence. A carboxyl terminus of PLAUR (located in the domain DIII) is linked to a glycosylphosphatidylinositol (GPI) anchor, which links PLAUR to a surface of a phospholipid bilayer of a cell membrane. An amino terminus of PLAUR (located in the domain DI) provides a binding site for the urokinase-type plasminogen activator (uPA), which can bind to uPA with high affinity. Under inflammatory stimulation, PLAUR detaches from a cell membrane under the action of various proteins to form a soluble urokinase-type plasminogen activator receptor (suPAR). Under different physiological and pathological states, a serum PLAUR concentration can reflect an inflammatory response state of a body and an activation degree of an immune system. Therefore, PLAUR was first known as an inflammatory marker. Studies in recent years have shown that PLAUR is also closely related to the occurrence and development of CVDs, such as atherosclerosis and heart failure. For example, since PLAUR is highly expressed in macrophages and there is a high PLAUR concentration in atheromatous plaques, it is speculated that PLAUR is related to an inflammatory response in atherosclerotic plaques, and the specific mechanism thereof needs to be further studied. Another study has shown that PLAUR is significantly positively correlated with an NT-proBNP level and a heart failure incidence. An elevated PLAUR level may indicate impaired microvascular circulation that will lead to impaired myocardial function, which is closely related to the occurrence of heart failure. At present, there is no report indicating that PLAUR is related to VHD worldwide.

### SUMMARY

In the present disclosure, serum PLAUR levels in VHD patients and healthy volunteers are clinically studied for the first time. Results show that PLAUR can be used as a biomarker for auxiliary diagnosis of VHD.

A study process is as follows:

1. Serum/plasma is collected from healthy volunteers or VHD patients by a conventional recognized method.

2. Serum N-terminal pro-brain natriuretic peptide (NT-ProBNP) levels in the healthy volunteers or VHD patients are determined with an NT-ProBNP assay kit (chemiluminescence method).

3. PLAUR levels in serum/plasma of the healthy volunteers or VHD patients are determined with an enzyme-linked immunosorbent assay (ELISA) kit.

Therefore, a technical solution of the present disclosure provides a marker (PLAUR) for auxiliary diagnosis of VHD, and the marker refers to serum PLAUR. With a reagent for detecting a PLAUR level in serum, a product for auxiliary diagnosis of VHD can be prepared. The product may be a kit, an array, or a test strip, or may be some other test products.

### BRIEF DESCRIPTION OF THE DRAWINGS

FIG. 1 is a technical flow chart of the present disclosure.

FIG. 2 shows the detection results of NT-ProBNP in serum of healthy volunteers and VHD patients.

FIG. 3 shows the detection results of hsCRP level in serum of healthy volunteers and VHD patients.

FIG. 4 shows the detection results of PLAUR in serum of healthy volunteers and VHD patients.

### DETAILED DESCRIPTION OF THE EMBODIMENTS

In order to make the technical problems, technical solutions, and advantages of the present disclosure clearer, the present disclosure is described in detail below with reference to specific examples.

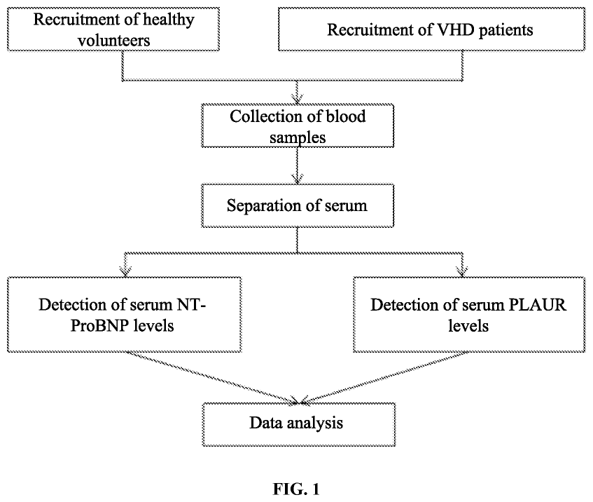

A technical flow of the present disclosure is shown in FIG. 1.

### Example 1 Collection of Serum from Healthy Volunteers or VHD Patients

1.1 Recruitment of healthy volunteers: Healthy volunteers were recruited and signed an informed consent form. Twenty-seven healthy volunteers including fourteen males and thirteen females were recruited for this study, where eight were between the ages of 21 to 30, eleven were between the ages of 31 to 40, three were between the ages of 41 to 50, and five were between the ages of 51 to 60.

1.2 Recruitment of VHD patients: The VHD patients recruited for this study were admitted to Nanhai Hospital of Guangdong Provincial People's Hospital, and blood was collected. This study was conducted with patients that voluntarily signed an informed consent form. The patients recruited for this study included twenty-three males and twenty-five females, where one was below the age of 20, five were between the ages of 31 to 40, nine were between the ages of 41 to 50, fifteen were between the ages 51 to 60, thirty were between the ages of 61 to 70, and three were above the age of 70.

1.3 Separation of serum: A blood collection tube was centrifuged at 1,300 g at room temperature for 10 min, and the serum at an upper layer was collected and added to a new centrifuge tube. The collected serum could be directly tested for the NT-ProBNP or PLAUR level or could be dispensed and stored at −80° C. for later use.

### Example 2 Detection of a Serum NT-ProBNP Level

2.1 Sample preparation: The freshly-separated serum could be directly tested. A cryopreserved sample must be thawed at room temperature and then fully mixed by low-speed vortexing or inverting.

2.2 Detection of an NT-ProBNP level: An NT-ProBNP level was detected according to instructions of an NT-ProBNP assay kit (chemiluminescence method) (Shenzhen New Industries Biomedical Engineering Co., Ltd. (SNIBE), 130206004M).

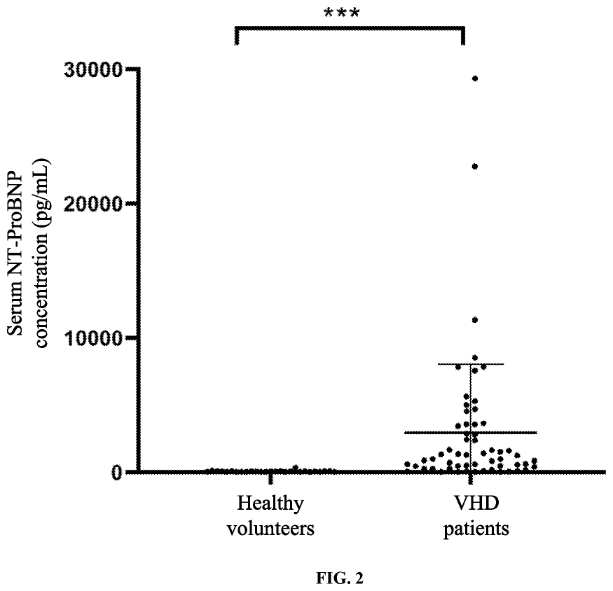

Result analysis: As shown in FIG. 2 and Table 1, the serum NT-ProBNP level in the VHD patient group was significantly higher than the serum NT-ProBNP level in the healthy volunteer group. However, NT-ProBNP levels in 34.5% ( 20/58) of the diagnosed patients were in a normal range, indicating that the NT-ProBNP level, when serving as a biomarker for VHD, exhibited undesirable sensitivity/accuracy. Therefore, the clinical evaluation with the single biomarker of NT-ProBNP is not complete, and multiple indexes must be combined to achieve an accurate determination.

### Example 3 Detection of a Serum hsCRP Level

3.1 Sample preparation: The freshly-separated serum could be directly tested. A cryopreserved sample must be thawed at room temperature and then fully mixed by low-speed vortexing or inverting.

3.2 Detection of an hsCRP level: An hsCRP level was detected according to instructions of the C-reactive protein assay kit (chemiluminescence method) (Shenzhen New Industries Biomedical Engineering Co., Ltd. (SNIBE), 130216002M).

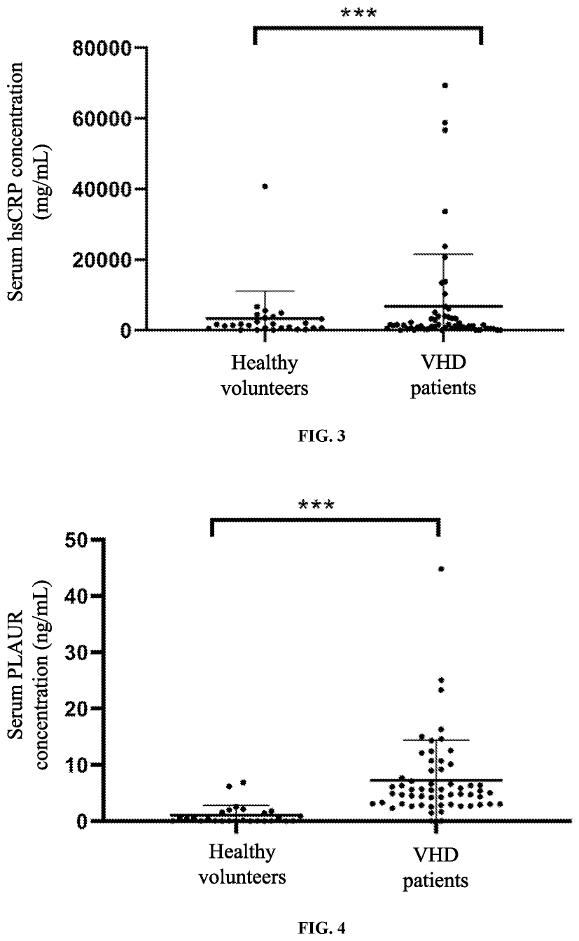

Result analysis: As shown in FIG. 3 and Table 1, serum hsCRP levels in 31.0% ( 18/58) of the diagnosed VHD patients were in the normal range, indicating that the hsCRP level, when serving as a biomarker for VHD, exhibited undesirable sensitivity/accuracy.

### Example 4 Detection of a Serum PLAUR Level

4.1 Sample preparation: The freshly-separated serum could be directly tested. A cryopreserved sample must be thawed and then centrifuged, and the resulting supernatant was collected for detection.

4.2 Detection of a PLAUR level: A PLAUR level was detected according to instructions of the human PLAUR/UPAR ELISA kit (CUSABIO, CSB-E04752h).

Result analysis: As shown in FIG. 4 and Table 1, an average serum PLAUR level (1.05 ng/mL) in the healthy volunteers was significantly lower than an average serum PLAUR level in the VHD patients, and PLAUR was not detected in about 44% of the healthy volunteers. With an average PLAUR level in the healthy population as an evaluation criterion, PLAUR levels in only 3.4% ( 2/58) of the VHD patients were lower than this criterion. Therefore, PLAUR can be used as a biomarker for auxiliary diagnosis of VHD, with much higher sensitivity/accuracy than NT-ProBNP and hsCRP.

TABLE 1
Serum PLAUR, NT-proBNP, and hsCRP levels in VHD patients

| | | | | | | | Whether it is | |
|---|---|---|---|---|---|---|---|---|
| | | | | Whether the | Whether | | higher than | |
| | | | | NT-proBNP | the hsCRP | Serum | an average | |
| | | | | level is in a | level is in | PLAUR | of the | |
| | Diagnosis and | | | normal | a normal | concentration | healthy | Times |
| No. | description | Age | Sex | range? | range? | (ng/mL) | people? | higher |
| 1 | MI | 37 | Male | Yes | Yes | 4.465 | Yes | 4.26 |
| 2 | MI | 53 | Male | Yes | Yes | 3.095 | Yes | 2.95 |
| 3 | MI | 67 | Female | No | Yes | 5.026 | Yes | 4.80 |
| 4 | MI | 64 | Female | No | No | 15.027 | Yes | 14.34 |
| 5 | MI | 59 | Male | No | Yes | 1.438 | Yes | 1.37 |
| 6 | MI | 57 | Male | No | No | 4.439 | Yes | 4.24 |
| 7 | MI | 46 | Male | No | No | 4.817 | Yes | 4.60 |
| 8 | MI | 54 | Female | No | No | 6.631 | Yes | 6.33 |
| 9 | MI | 54 | Male | No | No | 14.302 | Yes | 13.65 |
| 10 | MI | 70 | Female | Yes | Yes | 1.633 | Yes | 1.56 |
| 11 | MI | 37 | Male | Yes | No | 3.03 | Yes | 2.89 |
| 12 | MI | 68 | Male | Yes | No | 8.999 | Yes | 8.59 |
| 13 | MI | 63 | Female | No | No | 4.739 | Yes | 4.52 |
| 14 | MI | 68 | Male | Yes | Yes | 6.304 | Yes | 6.02 |
| 15 | MI | 65 | Female | No | No | 2.925 | Yes | 2.79 |
| 16 | MI | 66 | Female | Yes | Yes | 3.03 | Yes | 2.89 |
| 17 | MI | 52 | Male | Yes | No | 4.908 | Yes | 4.68 |
| 18 | MI | 71 | Male | Yes | No | 6.298 | Yes | 6.01 |
| 19 | MI | 53 | Female | Yes | No | 6.096 | Yes | 5.82 |
| 20 | MI | 62 | Female | No | No | 12.091 | Yes | 11.54 |
| 21 | MI | 48 | Male | No | Yes | 10.701 | Yes | 10.21 |
| 22 | MI | 38 | Female | Yes | Yes | 9.188 | Yes | 8.77 |
| 23 | AI, MI | 64 | Male | No | No | 2.938 | Yes | 2.80 |
| 24 | AI, MI | 62 | Female | Yes | No | 5.626 | Yes | 5.37 |
| 25 | AI, MI | 65 | Male | Yes | No | 10.127 | Yes | 9.66 |
| 26 | AI, MI | 65 | Female | No | No | 25.066 | Yes | 23.92 |
| 27 | AI, MI | 56 | Male | No | No | 7.087 | Yes | 6.76 |
| 28 | AI, MI, TI | 65 | Female | No | No | 5.632 | Yes | 5.37 |
| 29 | MI, TI | 48 | Female | No | Yes | 14.616 | Yes | 13.95 |
| 30 | MI, MS, heart failure | 51 | Female | No | No | 2.69 | Yes | 2.57 |
| 31 | MI, hyperaldosteronism | 60 | Female | No | Yes | 0 | No | 0.00 |
| 32 | MI, after coronary stenting | 77 | Female | No | No | 16.331 | Yes | 15.58 |
| 33 | MS, MI | 56 | Male | No | Yes | 2.69 | Yes | 2.57 |
| 34 | AF | 57 | Male | No | Yes | 2.297 | Yes | 2.19 |
| 35 | RHD, MS, MI, AS, AI | 64 | Female | No | No | 44.822 | Yes | 42.77 |
| 36 | RHD, MS | 61 | Female | No | No | 0 | No | 0.00 |
| 37 | RHD, MS, AF | 31 | Female | No | Yes | 7.654 | Yes | 7.30 |
| 38 | MI | 46 | Male | No | No | 5.703 | Yes | 5.44 |
| 39 | Paravalvular leak of mitral | 67 | Female | No | No | 23.305 | Yes | 22.24 |
| | valve bioprosthesis | | | | | | | |
| 40 | Mitral valve prolapse | 50 | Male | No | Yes | 3.054 | Yes | 2.91 |
| 41 | MS, AS | 63 | Male | Yes | No | 4.728 | Yes | 4.51 |
| 42 | Rheumatic mitral valve | 54 | Female | No | Yes | 3.33 | Yes | 3.18 |
| | disease | | | | | | | |
| 43 | Paravalvular leak of bivalve | 69 | Male | No | No | 10.712 | Yes | 10.22 |
| | and main valve | | | | | | | |
| 44 | AI | 38 | Male | No | No | 5.864 | Yes | 5.60 |
| 45 | AI | 73 | Male | Yes | Yes | 12.386 | Yes | 11.82 |
| 46 | AI | 68 | Male | No | No | 5.412 | Yes | 5.16 |
| 47 | AF | 64 | Male | Yes | No | 6.606 | Yes | 6.30 |
| 48 | AS | 69 | Male | Yes | No | 2.733 | Yes | 2.61 |
| 49 | AS | 59 | Female | No | No | 6.431 | Yes | 6.14 |
| 50 | AS | 65 | Female | No | No | 12.546 | Yes | 11.97 |
| 51 | AS, MS | 50 | Male | No | No | 2.835 | Yes | 2.71 |
| 52 | AI, dilatation of the | 61 | Male | Yes | No | 4.277 | Yes | 4.08 |
| | ascending aorta | | | | | | | |
| 53 | Dilated cardiomyopathy, | 46 | Male | No | No | 4.699 | Yes | 4.48 |
| | heart failure | | | | | | | |
| 54 | Coronary heart disease | 44 | Male | Yes | No | 5.514 | Yes | 5.26 |
| | (CHD) | | | | | | | |
| 55 | Heart failure | 47 | Female | No | No | 4.364 | Yes | 4.16 |
| 56 | CHD | 70 | Male | No | No | 2.835 | Yes | 2.71 |
| 57 | CHD | 18 | Male | Yes | Yes | 2.573 | Yes | 2.46 |
| 58 | CHD | 53 | Male | No | No | 4.233 | Yes | 4.04 |

Notes:
MI: mitral insufficiency; AI: aortic insufficiency; TI, tricuspid insufficiency; MS: mitral stenosis; RHD: rheumatic heart disease; AF: atrial fibrillation; and AS: aortic stenosis.

The present disclosure provides for the first time using PLAUR as a biomarker for the auxiliary diagnosis of VHD. When the PLAUR is used for the auxiliary diagnosis, the diagnostic result can be obtained in only one working day (high detection speed) with sensitivity/accuracy much higher than the sensitivity/accuracy of NT-ProBNP and hsCRP, and a high-throughput operation is enabled. Therefore, the serum PLAUR level can be used alone as a new diagnostic basis for VHD, which has higher accuracy than the existing markers. Of course, the serum PLAUR can also be used in combination with the existing biomarkers, such as BNP, to achieve the comprehensive diagnosis of VHD based on multiple detection results, thereby improving the diagnostic accuracy of VHD.

The above are preferred implementations of the present disclosure. It should be noted that a person of ordinary skill in the art may further make several improvements and modifications without departing from the principle of the present disclosure, but such improvements and modifications should also be deemed as falling within the protection scope of the present disclosure.

What is claimed is:

1. A use of a urokinase-type plasminogen activator receptor (PLAUR) in a preparation of a product for an auxiliary diagnosis of a valvular heart disease (VHD).

2. The use according to claim 1, wherein the product is used for detecting a PLAUR level.

3. The use according to claim 2, wherein a test object of the product is serum or plasma.

4. The use according to claim 1, wherein the product is a kit, an array, or a test strip.

5. The use according to claim 2, wherein the product is a kit, an array, or a test strip.

6. The use according to claim 3, wherein the product is a kit, an array, or a test strip.
